(19) United States
(12) Patent Application Publication
(10) Pub. No.: US 2004/0010023 A1
(43) Pub. Date: Jan. 15, 2004

(54) Biphenyl derivatives and the use thereof as integrin inhibitors

(76) Inventors: Wolfgang Stahle, Ingelheim (DE); Gunter Holzemann, Seeheim-Jugenheim (DE); Simon Goodman, Griesheim (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/362,234

(22) PCT Filed: Aug. 2, 2001
(86) PCT No.: PCT/EP01/08970

(30) Foreign Application Priority Data
Aug. 23, 2000 (DE) 100 41 423.0

(51) Int. Cl. A61K 31/4196; A61K 31/4172; A61K 31/4152; C07D 249/16; C07D 233/44; C07D 231/06

(52) U.S. Cl. 514/381; 514/383; 514/400; 548/257; 548/264.8; 548/326.5; 548/371.4; 548/557; 514/406; 514/426

(57) ABSTRACT

The invention relates to novel biphenyl derivatives of the general formula (I), wherein R⁴ represents an aromatic heterocycle, and to the physiologically acceptable salts or solvates thereof. The inventive compounds are integrin inhibitors and are used for combating thromboses, cardiac infarction, coronary heart diseases, arteriosclerosis, inflammations, tumors, osteoporosis, infections and restenosis following angioplasty or for pathological processes that are maintained or propagated by angiogenesis.

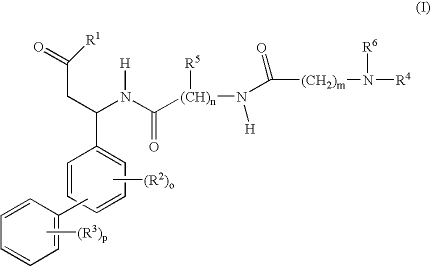

The invention relates to biphenyl derivatives of the formula I

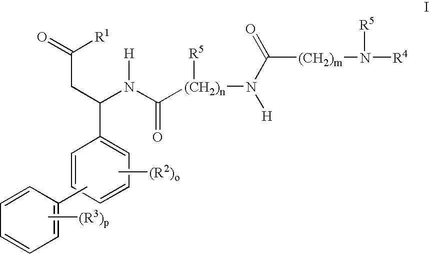

in which

R¹ is OR or N(R)₂,

R is H, A, cycloalkyl, Ar, arylalkyl or Pol,

R² and R³ in each case independently of one another are H, A, Hal, NO₂, OR, N(R)₂, CN, CO—R, SO₃R, SO₂R, NH—C(O)A or SR,

R⁴ is a mono- or bicyclic aromatic heterocycle having 1 to 4 N atoms, which can be mono- or disubstituted by Hal, R, OR, CN, N(R⁵)₂ or NO₂, where pyridine, pyridazine, pyrimidine, pyrazine, 1,3,5-, 1,2,4-, and 1,2,3-triazine and tetrazine are excluded,

R⁵ is H or A,

R⁶ is Hal or NO₂,

A is alkyl having 1 to 8 C atoms, where the alkyl groups can be mono- or polysubstituted by R⁶ and/or their alkyl carbon chain can be interrupted by —O—,

Ar is aryl which is unsubstituted or mono-, di- or trisubstituted,

cycloalkyl is cycloalkyl having 3 to 15 C atoms,

Hal is F, Cl, Br or I,

Pol is a solid phase without a terminal functional group,

n, m in each case independently of one another are 1, 2, 3, 4, 5 or 6,

o is 1, 2, 3 or 4,

p is 1, 2, 3, 4 or 5,

and their physioligically acceptable salts and solvates.

Compounds which in some cases are similar are disclosed in WO 97/26250.

The invention was based on the object of finding novel compounds having valuable properties, in particular those which can be used for the production of medicaments.

It has been found that the compounds of the formula I and their salts have very valuable pharmacological properties, together with good tolerability. They act especially as integrin inhibitors, where they inhibit, in particular, the interactions of the αvβ3 or αvβ5 integrin receptors with ligands, such as the binding of vitronectin to the αvβ3 integrin receptor. Integrins are membrane-bound, heterodimeric glycoproteins which consist of an ax subunit and a smaller β subunit. The relative affinity and specificity for ligand binding is determined by combination of the various α and β sub-units. The compounds according to the invention exhibit particular activity in the case of the integrins αvγ3, αvβ3, αvβ5, α11bβ3 and also αvβ6 and αvβ8, preferably of αvβ3, αvβ5 and αvβ6. In particular, potent selective inhibitors of the integrin αvβ3 have been found. The αvβ3 integrin is expressed on a number of cells, e.g. endothelial cells, cells of the vascular smooth musculature, for example of the aorta, cells for the degradation of bone matrix (osteoclasts) or tumour cells.

The action of the compounds according to the invention can be demonstrated, for example, by the method which is described by J. W. Smith et al. in J. Biol. Chem. 1990, 265, 12267-12271.

The dependence of the formation of angiogenesis on the interaction between vascular integrins and extracellular matrix proteins is described by P. C. Brooks, R. A. Clark and D. A. Cheresh in Science 1994, 264, 569-571.

The possibility of the inhibition of this interaction and thus the initiation of apoptosis (programmed cell death) of angiogenic vascular cells by a cyclic peptide is described by P. C. Brooks, A. M. Montgomery, M. Rosenfeld, R. A. Reisfeld, T. Hu, G. Klier and D. A. Cheresh in Cell 1994, 79, 1157-1164. In this, for example, αvβ3 antagonists or antibodies against αvβ3 were described which bring about shrinkage of tumours by initiation of apoptosis.

The experimental proof that the compounds according to the invention also prevent the attachment of living cells to the corresponding matrix proteins and accordingly also prevent the attachment of tumour cells to matrix proteins can be furnished in a cell adhesion test, analogously to the method of F. Mitjans et al., J. Cell Science 1995, 108, 2825-2838.

The compounds of the formula I can inhibit the binding of metalloproteinases to integrins and thus prevent the cells being able to utilize the enzymatic activity of the proteinase. An example can be found in the inhibitability of the binding of MMP-2 (matrix metallo-proteinase 2) to the vitronectin receptor αvβ3 by a cyclo-RGD peptide, as described in P. C. Brooks et al., Cell 1996, 85, 683-693.

Compounds of the formula I which block the interaction of integrin receptors and ligands, such as of fibrinogen to the fibrinogen receptor (glycoprotein IIb/IIIa), prevent, as antagonists, the spread of tumour cells by metastasis and can therefore be employed as substances having antimetastatic action in operations in which tumours are surgically removed or attacked. This is confirmed by the following observations:

The spread of tumour cells from a local tumour into the vascular system takes place by the formation of microaggregates (microthrombi) by the interaction of the tumour cells with blood platelets. The tumour cells are screened by the protection in the microaggregate and are not recognized by the cells of the immune system. The microaggregates can fix to vessel walls, whereby a further penetration of tumour cells into the tissue is facilitated. Since the formation of the microthrombi is mediated by ligand binding to the corresponding integrin receptors, e.g. αvβ3 or αIIbβ3, on activated blood platelets, the corresponding antagonists can be regarded as active metastasis inhibitors.

The action of a compound on an αvβ5 integrin receptor and thus the activity as an inhibitor can be demonstrated, for example, according to the method which is described by J. W. Smith et al. in J. Biol. Chem. 1990, 265, 12267-12271.

The compounds of the formula I can be employed as pharmaceutical active compounds in human and veterinary medicine, in particular for the prophylaxis and/or therapy of disorders of the circulation, thrombosis, cardiac infarcts, arteriosclerosis, apoplexy, angina pectoris, oncoses, such as tumour development or tumour metastasis, osteolytic diseases such as osteoporosis, pathologically angiogenic disorders such as inflammation, ophthalmological disorders, diabetic retinopathy, macular degeneration, myopia, ocular histoplasmosis, rheumatoid arthritis, osteoarthritis, rubeotic glaucoma, ulcerative colitis, Crohn's disease, atherosclerosis, psoriasis, restenosis after angioplasty, multiple sclerosis, viral infection, bacterial infection, fungal infection, in acute kidney failure and in wound healing for assisting the healing process.

αvβ6 is a relatively rare integrin (Busk et al., 1992 J. Biol. Chem. 267(9), 5790) which is formed in increased amounts in repair processes in the epithelial tissue and preferably binds the natural matrix molecules fibronectin and tenascin (Wang et al., 1996;. Am. J. Respir. Cell Mol. Biol. 15(5), 664). The physiological and pathological functions of αvβ6 are still not accurately known, but it is suspected that this integrin plays an important part in physiological processes and conditions (e.g. inflammation, wound healing, tumours) in which epithelial cells are involved. Thus αvβ6 is expressed on keratinocytes in wounds (Haapasalmi et al., 1996, J. Invest. Dermatol. 106(1,), 42), from which it can be assumed that in addition to wound-healing processes and inflammation other pathological occurrences in the skin, such as psoriasis, can also be influenced by agonists or antagonists of the said integrin. αvβ6 furthermore plays a part in the respiratory tract epithelium (Weinacker et al., 1995, Am. J. Respir. Cell Mol. Biol. 12(5), 547), so that corresponding agonists/antagonists of this integrin could be employed successfully in respiratory tract diseases, such as bronchitis, asthma, pulmonary fibroses and respiratory tract tumours. Finally, it is known that αvβ6 also plays a part in the intestinal epithelium, so that corresponding integrin agonists/antagonists could find use in the treatment of inflammation, tumours and wounds of the gastrointestinal tract.

The action of a compound on an αvβ6 integrin receptor and thus the activity as an inhibitor can be demonstrated, for example, by the method which is described by J. W. Smith et al. in J. Biol. Chem. 1990, 265, 12267-12271.

The compounds of the formula I can be employed as substances having antimicrobial action in operations where biomaterials, implants, catheters or heart pacemakers are used. They have an antiseptic action. The efficacy of the antimicrobial activity can be demonstrated by the procedure described by P. Valentin-Weigund et al., in Infection and Immunity, 1988, 2851-2855.

A measure of the absorption of a pharmaceutical active compound in an organism is its bioavailability.

If the pharmaceutical active compound is supplied intravenously to the organism in the form of an injection solution, its absolute bioavailability, i.e. the proportion of the pharmacon which passes unchanged into the systemic blood, i.e. into the general circulation, is 100%.

On oral administration of a therapeutic active compound, as a rule the active compound is present in the formulation as a solid and must therefore first be dissolved so that it can overcome the entry barriers, for example the gastrointestinal tract, the oralmucous, membrane, nasal membranes or the skin, in particular the stratum corneum, or can be absorbed by the body. Data on the pharmacokinetics, i.e. on the bioavailability, can be obtained analogously to the method of J. Shaffer et al., J. Pharm. Sciences, 1999, 88, 313-318.

A further measure of the absorbability of a therapeutic active compound is the logD value, since this value is a measure of the lipophilicity of a molecule.

The compounds of the formula I have at least one chiral centre and can therefore occur in a number of stereoisomeric forms. All these forms (e.g. D and L forms) and their mixtures (e.g. the DL forms) are included in the formula.

The compounds according to the invention according to claim 1 also include ‘prodrug derivatives’, i.e. compounds of-the formula I modified with, for example, alkyl or acyl groups, sugars or oligopeptides, which are rapidly cleaved to the active compounds according to the invention in the organism.

Free amino groups or free hydroxyl groups as substituents of the compounds of the formula I can furthermore be provided with appropriate protective groups.

Solvates of the compounds of the formula I are understood as meaning adducts of inert solvent molecules to the compounds of the formula I, which are formed on account of their mutual attractive force. Solvates are, for example, mono- or dihydrates or addition compounds with alcohols, such as with methanol or ethanol.

The invention relates to the compounds of the formula I and their salts and solvates according to claim 1, and to a process for the preparation of compounds of the formula I and its salts and solvates, characterized in that

(a) a compound of the formula II

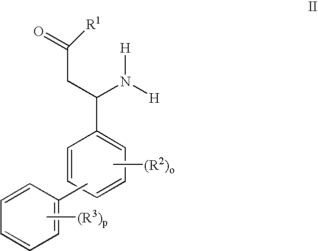

in which R, R¹, R², R³, o and p have the meanings indicated in claim 1, but R≠H and in which free hydroxyl or amino groups as substituents R² or R³ are present protected by protective groups, is reacted with a compound of the formula III

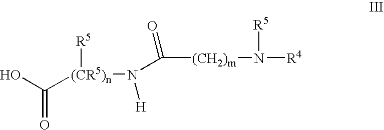

in which R⁴, R⁵, n and m have the meanings indicated in claim 1

and, if appropriate, the radical R≠H is converted into the radical R=H and the protective groups on R² and/or R³ are removed, or

(b) a compound of the formula IV

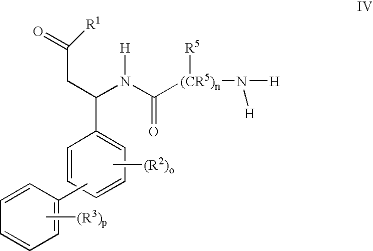

in which R, R¹, R², R³, R⁵, n, o and p have the meanings indicated in claim 1, but R≠H and in which free hydroxyl or amino groups as substituents R² or R³ are present protected by protective groups,

is reacted with a compound of the formula V

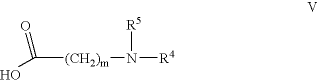

in which R⁴, R⁵ and m have the meanings indicated in claim 1

and, if appropriate, the radical R≠H is converted into the radical R=H and the protective groups on R² and/or R³ are removed, or

(c) in a compound of the formula I, one or more radicals R, R¹, R², R³, R⁴ and/or R⁵ are converted into one or more radicals R, R¹, R², R³, R⁴ and/or R⁵, by, for example, i) alkylating a hydroxyl group,

ii) hydrolysing an ester group to a carboxyl group,

iii) esterifying a carboxyl group,

iv) alkylating an amino group,

v) reacting an aryl bromide or iodide by means of a Suzuki coupling with boronic acids to give the corresponding coupling products, or

vi) acylating an amino group, and/or

converting a basic or acidic compound of the formula I into one of its salts or solvates by treating with an acid or base.

In the above formulae, A is alkyl, is linear or branched, and has 1 to 8, preferably 1, 2, 3, 4, 5 or 6 C atoms. A is preferably methyl, furthermore ethyl, n-propyl, isopropyl, n-butyl, sec-butyl or tert-butyl, furthermore also pentyl, 1-, 2-, or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, heptyl or octyl. Furthermore preferred embodiments of A are the alkyl groups mentioned, which, however, can be mono- or polysubstituted by Hal or NO₂, preferably trifluoromethyl, 2,2,2-trifluoroethyl or 2-nitroethyl, or alkyl groups whose carbon chain can be interrupted by —O—, preferably —CH₂—O—CH₃, —CH₂—O—CH₂—CH₃ or —CH₂—CH₂—O—CH₃.

Methyl or ethyl is particularly preferred for A.

Ar is aryl which is unsubstituted or mono-, di- or trisubstituted by A, CF₃, OH, OA, OCF₃, CN, NO₂ or Hal, where aryl is phenyl, naphthyl, anthryl or biphenylyl. Ar is preferably phenyl or naphthyl, which is unsubstituted or mono-, di- or trisubstituted by A, CF₃, OH, OA, OCF₃, CN, NO₂ or Hal. Ar is particularly preferably phenyl.

Arylalkyl is —(CH₂)ₓ—Ar, where Ar has one of the preferred meanings indicated previously, and where x can be 1, 2 or 3. Arylalkyl is preferably benzyl, phenylethyl or phenylpropyl; benzyl is particularly preferred for arylalkyl.

Cycloalkyl having 3 to 15 C atoms is preferably cyclopropyl, cyclobutyl, cyclopentyl, cyclohexyl, cycloheptyl or cyclooctyl. Cycloalkyl istalso mono- or bicyclic terpenes, preferably p-menthane, menthol, pinane, bornane or camphor, where each known stereoisomeric form is included, or adamantyl. For camphor, this means both L-camphor and D-camphor.

Hal is preferably F, Cl or bromine. Hal is particularly preferably F or Cl.

Pol is a solid phase without a terminal functional group, such as explained in greater detail below. The terms solid phase and resin are used synonymously below.

In the biphenyl derivatives of the formula I, the second phenyl radical is preferably coupled to the first phenyl radical in the 3 or 4 position, particularly preferably on the 4 position of the first phenyl ring.

R¹ is OR or N(R)₂, where R has one of the meanings below. R¹ is particularly preferably OH.

R is H, A, cycloalkyl, Ar, arylalkyl or Pol, where A, cycloalkyl, Ar and arylalkyl have one of the previously described meanings and Pol has one of the meanings described below. R is particularly preferably Pol or H. R is very particularly preferably H.

R² and R³ are, in each case independently of one another, H, A, Hal, NO₂, OR, N(R)₂, CN, CO—R, SO₃R, SO₂R, NNH—C(O)A or SR, where A and R have one of the previously described meanings. R² is particularly preferably H. R³ is particularly preferably Hal, OA or CN; R³ is very particularly preferably Hal.

R⁴ is preferably substituted or unsubstituted 1-, 2- or 3-pyrrolyl, 1-, 2-, 4- or 5-imidazolyl, 3-, 4- or 5-pyrazolyl, furthermore preferably 1-, 2-, 3-, 4-, 5-, 6- or 7-1H-indolyl, 1-, 2-, 4- or 5-benzimidazolyl, 1-, 3-, 4-, 5-, 6- or 7-benzopyrazolyl, 1-, 2-, 3-, 4-, 5-, 6-, 7- or 8-quinolinyl, 1-, 3-, 4-, -5-, 6-, 7- or 8-isoquinolinyl, 3-, A4-, 5-, 6-, 7- or 8-cinnolinyl, 1-, 4-, 5-, 6-, 7- or 8-phthalazinyl, 2-, 3-, 5-, 6-, 7- or 8-quinoxalinyl, 2-, 4-, 5-, 6-, 7- or 8-quinazolinyl.

The heterocyclic radicals can also be partly or completely hydrogenated. Het¹ can thus also be 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -3-pyrrolyl, tetrahydro-1, -2- or -4-imidazolyl, 2,3-dihydro-1-, -2-, -3-, -4-, -5-, -6-, -7-1H-indolyl, 2,3-dihydro-1-, -2-, -3-, -4- or -5-pyrazolyl, tetrahydro-1-, -3- or -4-pyrazolyl, 1,5-dihydroimidazol-4-on-2- or -5-yl, 1,4-dihydro-1-, -2-, -3- or -4-pyridyl, 1,2,3,4-tetrahydro-1-, -2-, -3-, -4-, -5- or -6-pyridyl, 1,2,3,6-tetrahydro-1-, -2-, -3-, -4-, -5- or -6-pyridyl, 1-, 2-, 3- or 4-piperidinyl, 1-, 2-, 3- or 4-azepanyl, tetrahydro-2-, -3- or -4-pyranyl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolinyl, 1,2,3,4-tetrahydro-1-, -2-, -3-, -4, -5-, -6-, -7- or -8-isoquinolinyl.

The heterocyclic rings mentioned can also be mono- or disubstituted by ═O or NHA.

R⁴ is particularly preferably benzimidazol-2-yl, imidazol-2-yl, 4,5-dihydroimidazol-2-yl or 4,5-dihydro-5-oxoimidazol-2-yl; very particularly preferably benzimidazol-2-yl.

R⁵ is H or A, where A has one of the meanings previously indicated. R⁵ is particularly preferably H.

R⁶ is Hal or NO₂, where Hal has one of the meanings previously indicated. R⁶ is particularly preferably Hal.

m and n are in each case independently of one another 1, 2, 3, 4, 5 or 6, m is particularly preferably 1, 2, 3 or 4, m is very particularly preferably 3.

n is preferably 1 or 2, n is particularly preferably 1.

o is 1, 2, 3 or 4, particularly preferably 1.

p is 1, 2, 3, 4 or 5, particularly preferably 1 or 2.

Accordingly, the invention relates in particular to those compounds of the formula I in which at least one of the radicals mentioned has one of the preferred meanings indicated above. Some preferred groups of compounds can be expressed by the following subformulae Ia to Ic, which correspond to the formula I and in which the radicals not designated in greater detail have the meaning indicated in the formula I, but in which

| | | |
|---|---|---|
| in Ia | R¹ | is OR, |
| in Ib | R¹ | is OR and |
| | R | is H or A, |
| in Ic | R¹ | is OR, |
| | R | is H and |
| | R⁴ | is imidazol-2-yl or benzimidazol-2-yl. |

The compounds of the formula I according to claim 1 and also the starting substances for their preparation are otherwise prepared by methods known per se, such as are described in the literature (e.g. in the standard works such as Houben-Weyl, Methoden der organischen Chemie [Methods of Organic Chemistry], Georg-Thieme-Verlag, Stuttgart), namely under reaction conditions which are known and suitable for the reactions mentioned. Use can also be made in this case of variants which are known per se, but not mentioned here in greater detail.

The starting substances, if desired, can also be formed in situ such that they are not isolated from the reaction mixture, but immediately reacted further to give the compounds of the formula I according to claim 1.

A number oft—identical or different—protected amino and/or hydroxyl groups can also be present in the molecule of the starting substance. If the protective groups present are different from one another, in many cases they can be removed selectively (see for this: T. W. Greene, P. G. M. Wuts, Protective Groups in Organic Chemistry, 2^{nd} Ed., Wiley, New York 1991 or P. J. Kocienski, Protecting Groups, 1^{st} Ed., Georg Thieme Verlag, Stuttgart-New York, 1994).

The expression “amino protective group” is generally known and relates to groups which are suitable for protecting (or blocking) an amino group from chemical reactions. Typical groups of this type are, in particular, unsubstituted or substituted acyl, aryl, aralkoxymethyl or aralkyl groups. As the amino protective groups are removed after the desired reaction (or reaction sequence), their nature and size is otherwise not critical; however, those having 1-20, in particular 1-8, C atoms are preferred. The expression “acyl group” is to be interpreted in the widest sense in connection with the present process. It includes acyl groups and in particular alkoxycarbonyl, alkenyloxycarbonyl, aryloxycarbonyl and especially aralkoxycarbonyl groups derived from aliphatic, araliphatic, alicyclic, aromatic or heterocyclic carboxylic acids or sulfonic acids. Examples of acyl groups of this type are alkanoyl such as acetyl, propionyl, butyryl; aralkanoyl such as phenylacetyl; aroyl such as benzoyl or toluyl; aryloxyalkanoyl such as phenoxyacetyl; alkoxycarbonyl such as methoxycarbonyl, ethoxycarbonyl, 2,2,2-trichloroethoxy-carbonyl, BOC, 2-iodoethoxycarbonyl; alkenyloxycarbonyl such as allyloxycarbonyl (Aloc), aralkyloxycarbonyl such as CBZ (synonymous with Z), 4-methoxybenzyloxy-carbonyl (MOZ), 4-nitrobenzyloxycarbonyl or 9-fluorenylmethoxycarbonyl (Fmoc); 2-(phenylsulfonyl)-ethoxycarbonyl; trimethylsilylethoxycarbonyl (Teoc) or arylsulfonyl such as 4-methoxy-2,3,6-trimethylphenyl-sulfonyl (Mtr). Preferred amino protective groups are BOC, Fmoc and Aloc, furthermore CBZ, benzyl and acetyl. Particularly preferred protective groups are BOC and Fmoc.

The expression “hydroxyl protective group” is also generally known and relates to groups which are suitable for protecting a hydroxyl group from chemical reactions. Typical groups of this type are the abovementioned unsubstituted or substituted aryl, aralkyl, aroyl or acyl groups, furthermore also alkyl groups, alkyl-, aryl- or aralkylsilyl groups or O,O- or O,S-acetals. The nature and size of the hydroxyl protective groups is not critical, as they are removed again after the desired chemical reaction or reaction sequence; groups having 1-20, in particular 1-10, C atoms are preferred. Examples of hydroxyl protective groups are, inter alia, aralkyl groups such as benzyl, 4-methoxybenzyl or 2,4-dimethoxybenzyl, aroyl groups such as benzoyl or p-nitrobenzoyl, acyl groups such as acetyl or pivaloyl, p-toluenesulfonyl, alkyl groups such as methyl or tert-butyl, but also allyl, alkylsilyl groups such as trimethylsilyl (TMS), triisopropylsilyl (TIPS), tert-butyldimethylsilyl (TBS) or triethylsilyl, trimethylsilylethyl, aralkylsilyl groups such as tert-butyldiphenylsilyl (TBDPS), cyclic acetals such as isopropylidene, cyclopentylidene, cyclohexylidene, benzylidene, p-methoxybenzylidene or o,p-dimethoxybenzylidene acetal, acyclic acetals such as tetrahydropyranyl (Thp), methoxymethyl (MOM), methoxyethoxymethyl (MEM), benzyloxymethyl (BOM) or methylthiomethyl (MTM). Particularly preferred hydroxyl protective groups are benzyl, acetyl, tert-butyl or TBS.

The liberation of the compounds of the formula I from their functional derivatives is known from the literature for the protective group used in each case (e.g. T. W. Greene, P. G. M. Wuts, Protective Groups in Organic Chemistry, 2^{nd} Ed., Wiley, New York 1991 or P. J. Kocienski, Protecting Groups, 1^{st} Ed., Georg Thieme Verlag, Stuttgart-New York, 1994). Use can also be made in this case of variants which are known per se, but not mentioned here in greater detail.

The groups BOC and O-tert-butyl can be removed, for example, preferably using TFA in dichloromethane or using approximately 3 to 5 N HCl in dioxane at 15-30° C., the Fmoc group using an approximately 5 to 50% solution of dimethylamine, diethylamine or piperidine in DMF at 15-30° C. The Aloc group can be removed gently under noble metal catalysis in chloroform at 20-30° C. A preferred catalyst is tetrakis(triphenylphosphine)-palladium(0).

As a rule, the starting compounds of the formulae II to V and 1 to 3 are known. If they are novel, they can, however, be prepared by methods known per se.

The compounds of the formula I can also be synthesized on solid phase, the binding to the solid phase taking place to R¹. In the case of synthesis on solid phase, R¹ is also OPol, NHPol or NRPol, where Pol is a solid phase without a terminal functional group. Pol is representative of the polymeric support material and of all atoms of the anchor group of a solid phase, except for the terminal functional group. The anchor groups of a solid phase, also called linkers, are necessary for the binding of the compound to be functionalized to the solid phase. A summary of syntheses on solid phase and the solid phases and/or linkers which can be employed for this is given, for example, in Novabiochem—The Combinatorial Chemistry Catalog, March 99, pages S1-S72.

Particularly suitable solid phases for the synthesis of compounds according to the invention where R¹=OR are solid phases having a hydroxyl group as a terminal functionality, for example the Wang resin or polystyrene A OH. Particularly suitable solid phases for the synthesis of the compounds according to the invention where R¹=N(R)₂ are solid phases having an amino group as a terminal functionality, for example Rink amide resin.

Compounds of the formula II where R¹=OL, where L is Pol or R and R≠H, are prepared, for example, by the following reaction scheme 1, where SG₁ is an amino protective group, as described previously.

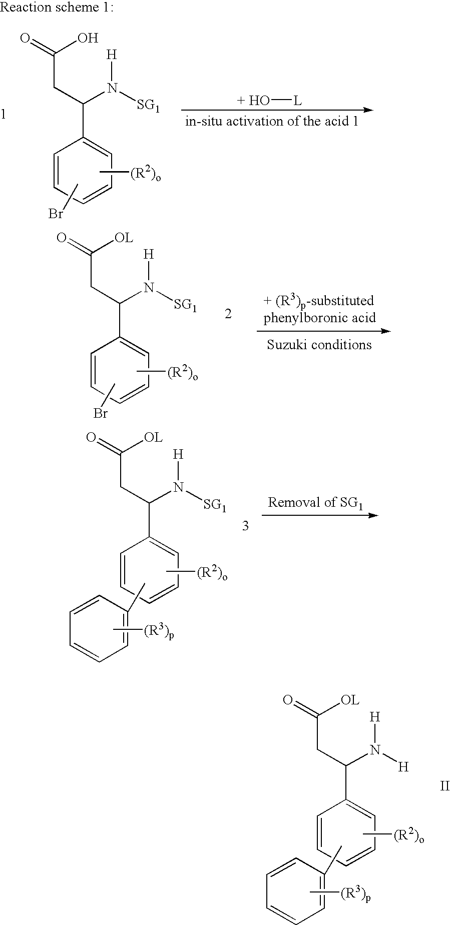

The bromophenyl-substituted carboxylic acid 1 is 10 activated in situ according to known methods, for example by reaction with diisopropylcarbodiimide, and reacted with the alcohol HO-L, where L has the meaning indicated above. The subsequent coupling of the compound 2 to an (R³)-substituted phenylboronic acid under Suzuki conditions produces the biphenyl derivative 3. The removal of the protective group SG under known conditions liberates a compound of the formula II.

The Suzuki reaction is expediently carried out under palladium-mediated conditions, preferably by addition of Pd(PPh₃)₄, in the presence of a base such as potassium carbonate in an inert solvent or solvent mixture, e.g. DMF at temperatures between 0° and 150°, preferably between 600 and 120°. Depending on the conditions used, the reaction time is between a few minutes and a number of days. The boronic acid derivatives can be prepared according to conventional methods or are commercially obtainable. The reactions can be carried out in analogy to the methods indicated in Suzuki et al., J. Am. Chem. Soc. 1989, 111, 314ff and in Suzuki et al. Chem. Rev. 1995, 95, 2457ff.

Compounds of the formula I are obtained by a peptide-analogous coupling of the compounds of the formula II to a compound of the formula III or by peptide-analogous coupling of the compounds of the formula IV to a compound of the formula. V under standard conditions. Compounds of the formula III are obtained by peptide-analogous coupling of a compound of the formula V to an amino compound H₂N—[C(R⁵)₂]ₙ—COOSG² under standard conditions, where SG² is a hydroxyl protective group such as described previously, which is removed after the coupling. Compounds of the formula IV are obtained by peptide-analogous coupling of a compound of the formula II to a carboxyl compound HOOC-[C(R⁵)₂]ₙ—NHSG₁ under standard conditions, where SG₁ is an amino protective group such as described previously, which is removed after the coupling.

Customary methods of peptide synthesis are described, for example, in Houben-Weyl, l.c., Volume 15/II, 1974, pages 1 to 806.

The coupling reaction is preferably carried out in the presence of a dehydrating agent, e.g. of a carbodiimide such as dicyclohexylcarbodiimide (DCC), N-(3-dimethyl-aminopropyl)-N′-ethylcarbodiimide hydrochloride (EDC) or diisopropylcarbodiimide (DIC), furthermore, for example, propanephosphonic anhydride (cf. Angew. Chem. 19890, 92, 129), diphenylphosphoryl azide or 2-ethoxy-N-ethoxycarbonyl-1,2-dihydroquinoline, in an inert solvent, e.g. a halogenated hydrocarbon such as dichloromethane, an ether such as tetrahydrofuran or dioxane, an amide such as DMF or dimethylacetamide, a nitrile such as acetonitrile, in dimethyl sulfoxide or in the presence of these solvents, at temperatures between approximately −10 and 40°, preferably between 0 and 30°. Depending on the conditions used, the reaction time is between a few minutes and a number of days. The addition of the coupling reagent TBTU (O-(benzotriazol-1-yl)-N,N,N′,N′-tetramethyluronium tetrafluoroborate)or O-(benzotriazol-1-yl)-N,N,N′,N′-tetramethyluronium hexafluorophosphate has proved to be particularly advantageous, as in the presence of one of these compounds only a small racemization occurs and no cytotoxic by-products are formed.

Instead of compounds of the formulae III and/or V, derivatives of compounds of the formulae III and/or V, preferably a preactivated carboxylic acid, or a carboxylic acid halide, a symmetrical or mixed anhydride or an active ester, can also be employed. Radicals of this type for the activation of the carboxyl group in typical acylation reactions are described in the literature (e.g. in the standard works such as Houben-Weyl, Methoden der organischen Chemie, Georg-Thieme-Verlag, Stuttgart). Activated esters are expediently formed in situ, e.g. by addition of HOBt (1-hydroxybenzotriazole) or N-hydroxysuccinimide.

As a rule, the reaction is carried out in an inert solvent, when using a carboxylic acid halide in the presence of an acid-binding agent, preferably of an organic base, such as triethylamine, dimethylaniline, pyridine or quinoline.

The addition of an alkali metal or alkaline earth metal hydroxide, carbonate or bicarbonate or of another salt of a weak acid of the alkali metals or alkaline earth metals, preferably of potassium, sodium, calcium or caesium, can also be favourable.

A base of the formula I can be converted into the associated acid addition salt using an acid, for example by reaction of equivalent amounts of base and the acid in an inert solvent such as ethanol and subsequent evaporation. For this reaction, suitable acids are in particular those which yield physiologically acceptable salts. Thus inorganic acids can be used, e.g. sulfuric acid, sulfurous acid, dithionic acid, nitric acid, hydrohalic acids such as hydrochloric acid or hydrobromic acid, phosphoric acids such as orthophosphoric acid, sulfamic acid, furthermore organic acids, in particular aliphatic, alicyclic, araliphatic, aromatic or heterocyclic mono- or polybasic carboxylic, sulfonic or sulfuric acid, e.g. formic acid, acetic acid, propionic acid, hexanoic acid, octanoic acid, decanoic acid, hexadecanoic acid, octadecanoic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, benzenesulfonic acid, trimethoxy-benzoic acid, adamantanecarboxylic acid, p-toluene-sulfonic acid, glycolic acid, embonic acid, chlorophenoxyacetic acid, aspartic acid, glutamic acid, proline, glyoxylic acid, palmitic acid, parachlorophenoxyisobutyric acid, cyclohexanecarboxylic acid, glucose 1-phosphate, naphthalenemono- and disulfonic acids or laurylsulfuric acid. Salts with physiologically unacceptable acids, e.g. picrates, can be used for the isolation and/or purification of the compounds of the formula I. On the other hand, compounds of the formula I can be converted using bases (e.g. sodium or potassium hydroxide or carbonate) into the corresponding metal salts, in particular alkali metal or alkaline earth metal salts, or into the corresponding ammonium salts.

The invention also relates to the compounds of the formula I according to claim 1 and their physiologically acceptable salts or solvates as pharmaceutical active compounds.

The invention furthermore relates to compounds of the formula I according to claim 1 and their physiologically acceptable salts or solvates as integrin inhibitors.

The invention also relates to the compounds of the formula I according to claim 1 and their physiologically acceptable salts or solvates for use in the control of diseases.

The invention furthermore relates to pharmaceutical preparations comprising at least one compound of the formula I and/or one of their physiologically acceptable salts or solvates which are prepared, in particular, in non-chemical ways. The compounds of the formula I here can be brought into a suitable dose form together with at least one solid, liquid and/or semi-liquid vehicle or excipient and if appropriate in combination with one or more further active compounds.

These preparations can be used as medicaments in human or veterinary medicine. Possible vehicles are organic or inorganic substances which are suitable for enteral (e.g. oral) or parenteral administration or topical application and do not react with the novel compounds, for example water, vegetable oils, benzyl alcohols, alkylene glycols, polyethylene glycols, glyceryl triacetate, gelatin, carbohydrates such as lactose or starch, magnesium stearate, talc and petroleum jelly. In particular, tablets, pills, coated tablets, capsules, powders, granules, syrups, juices or drops are used for oral administration, suppositories are used for rectal administration, solutions, preferably oily or aqueous solutions, furthermore suspensions, emulsions or implants, are used for parenteral administration, and ointments, creams or powders are used for topical application. The novel compounds can also be lyophilized and the lyophilizates obtained used, for example, for the production of injection preparations. The preparations indicated can be sterilized and/or can contain excipients such as lubricants, preservatives, stabilizing agents and/or wetting agents, emulsifiers, salts for influencing the osmotic pressure, buffer substances, colourants, flavourings and/or [lacuna] more further active compounds, e.g. one or more vitamins.

For administration as an inhalation, sprays can be used which contain the active compound either dissolved or suspended in a propellant or propellant mixture (e.g. CO₂ or chlorofluorocarbons). Expediently, the active compound is used here in micronized form, where one or more additional physiologically tolerable solvents can be present, e.g. ethanol. Inhalation solutions can be administered with the aid of customary inhalers.

The compounds of the formula I and their physiologically acceptable salts or solvates can be used as integrin inhibitors in the control of diseases, in particular of thromboses, cardiac infarcts, coronary heart diseases, arteriosclerosis, tumours, osteoporosis, inflammation and infections.

The compounds of the formula I according to claim 1 and/or their physiologically acceptable salts are also used in pathological processes which are supported or propagated by angiogenesis, in particular in tumours, restenoses, diabetic retinopathy or rheumatoid arthritis.

As a rule, the substances according to the invention are administered here in analogy to the compounds described in WO 97/26250, preferably in doses of between approximately 0.5 and 500 mg, in particular between 0.5 and 100 mg per dose unit. The daily dose is preferably between approximately 0.01 and 2 mg/kg of body weight. The specific dose for each patient depends, however, on all sorts of factors, for example on the efficacy of the specific compound employed, on the age, body weight, general state of health, sex, on the diet, on the time and route of administration, and on the excretion rate, pharmaceutical combination and severity of the particular disease to which the therapy relates. Parenteral administration is preferred.

The compounds of the formula I can furthermore be used as integrin ligands for the preparation of columns for affinity chromatography for the preparation of integrins in pure form. The ligand, i.e. a compound of the formula I, is here covalently coupled to a polymeric support via an anchor function, e.g. the carboxyl group.

Suitable polymeric support materials are the polymeric solid phases having preferably hydrophilic properties, which are known per se in peptide chemistry, for example crosslinked polysugars such as cellulose, sepharose or Sephadex^{R}, acrylamides, polymers based on polyethylene glycol or Tentakel polymer^{R}.

The preparation of the materials for affinity chromatography for integrin purification is carried out under conditions such as are customary and known, per se for the condensation of amino acids.

The compounds of the formula I contain one or more chiral centres and can therefore be present in racemic or in optically active form. Racemates obtained can be separated mechanically or chemically into the enantiomers by methods known per se. Preferably, diastereomers are formed from the racemic mixture by reaction with an optically active resolving agent. Suitable resolving agents are, for example, optically active acids, such as the D and L forms of tartaric acid, diacetyltartaric acid, dibenzoyltartaric acid, mandelic acid, malic acid, lactic acid or the various optically active camphorsulfonic acids such as camphorsulfonic acid. Separation of enantiomers with the aid of a column packed with an optically active resolving agent (e.g. dinitrobenzoylphenylglycine) is also advantageous; suitable eluents are, for example, a hexane/isopropanol/acetonitrile mixture, e.g. in the volume ratio 82:15:3.

Of course, it is also possible to obtain optically active compounds of the formula I by the methods described above by using starting compounds which are already optically active.

Above and below, all temperatures are indicated in 0° C. In the following examples, “customary working-up” means: if necessary, water is added, the mixture is adjusted to a pH of between 2 and 10, if necessary, depending on the constitution of the final product, and extracted with ethyl acetate or dichloromethane, the organic phase is separated off, dried over sodium sulfate and evaporated, and the residue is purified by chromatography on silica gel, by preparative HPLC and/or by crystallization. The purified compounds are optionally freeze-dried.

RT=retention time (in minutes) on HPLC in the following systems:

Column: Lichrosorb RP Select B 250×4 mm².

Eluents used are gradients of acetonitrile (B) with 0.08% TFA (trifluoroacetic acid) and water (A) with 0.1% TFA. The gradient is indicated in percent by volume of acetonitrile.

Preferred gradient: linear, t=0 min, A:B=80:20, t=15 min, A:B.=0:100 (t=time).

Detection at 225 nm.

The compounds purified by preparative HPLC are isolated as trifluoroacetates.

Mass spectrometry (MS) by means of FAB (fast atom bombardment): MS-FAB (M+H)⁺.

### EXAMPLE 1

(1) 4.168 g of diisopropylcarbodiimide (DIC) and 14.100 g of the solid phase polystyrene A OH (Rapp, item No. HA 1 400 00) are added to a solution of 11.4 g of 3-(4-bromophenyl)-3-tert-butoxycarbonylamino-propionic acid in 100 ml of N,N-dimethylformamide and treated with 100 mg of dimethylaminopyridine (DMAP). The reaction mixture is stirred at room temperature for 12 hours and then filtered off. The resin is washed three times with 150 ml each of DMF, dichloromethane and diethyl ether and dried. The resin-bound compound “AB” is obtained, Pol being the solid phase polystyrene A OH, without the functional OH group.

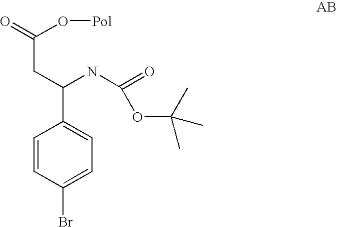

(2) 250 mg of tetrakis(triphenylphosphine)palladium(0) and 1.7 g of 4-dhlorophenylboronic acid are added under an inert gas atmosphere to a suspension of 5 g of the compound “AB” in 40 g of ethylene glycol dimethyl ether. The mixture is heated for 12 h at boiling temperature. After cooling the reaction mixture, 100 ml of a 25% ammonium acetate solution are added and the resin is filtered off. The resin is then washed with 50 ml of the following solvents or acids in each case: twice with dimethoxyethane (DME), once with water, once with 0.2 N hydrochloric acid, twice with DME, twice with dichloromethane and twice with methanol. Resin-bound 3-tert-butoxycarbonylamino-3-(4′-chlorobiphenyl-4-yl)propionic acid “BC” is obtained.

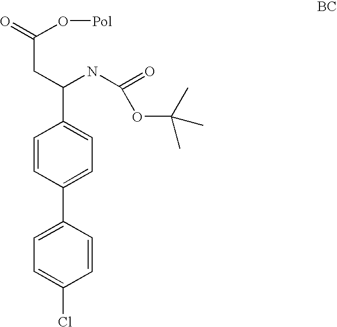

(3) 5 ml of trifluoroacetic acid are added to a suspension of 1 g of the solid phase “BC” in 5 ml of dichloromethane and the mixture is stirred for 30 min to remove the amino protective group. The resin is filtered and washed with dichloromethane and then treated with 10 ml of dimethylformamide (DMF). 0.7 g of DIC, 1.7 g of FMOC-protected glycine and 20 mg of DMAP are added to this suspension and it is stirred for 4-5 h. The resin is filtered off and washed with DMF, dichloromethane and methanol. Resin-bound 3-(4′-chloro-biphenyl-4-yl)-3-[2-(9H-fluoren-9-ylmethoxycarbonyl-amino)acetylamino]propionic acid “CD” is obtained.

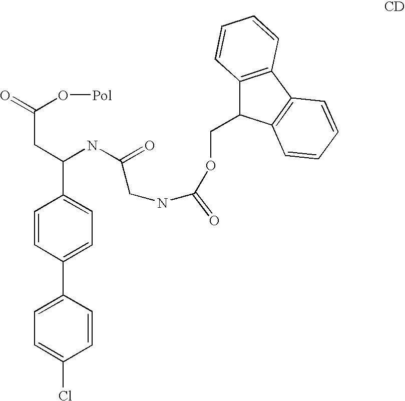

(4) A solution of 2.7 g of 1,1-thiocarbonyldiimidazole and 210 mg of imidazole in 20 ml of acetonitrile is cooled to 0° C. 1.7 g of ethyl 4-aminobutanoate hydrochloride and a solution of 1 g of triethylamine in 10 ml of acetonitrile are added. The mixture is allowed to thaw and is stirred for 3 h at room temperature. 2.2 g of 1,2-phenylenediamine are then added and the mixture is stirred for 3 h at 50° C. and 12 h at room temperature. The solvent is then distilled off and the residue is taken up in 20 ml of ethanol. 1.5 g of mercury(II) oxide and 23 mg of sulfur are added to this solution and it is heated to reflux for about 24 h. The solution is filtered and worked up in the customary manner.

Ethyl 4-(1H-benzimidazol-2-ylamino)butanoate is obtained; RT 7.09 min, FAB-MS (M+H)⁺ 248.

1 ml of 4 NaOH is added to a solution of 1.1 g of ethyl 4-(1H-benzimidazol-2-ylamino)butanoate in 30 ml of ethylene glycol monoethyl ether and the mixture is stirred for 6 h at 60° C. The solvent is distilled off and the residue is adjusted to pH 4 using 1N hydrochloric acid. The reaction mixture is then evaporated to dryness in vacuo. 4-(1H-Benzimidazol-2-ylamino)butanoic acid is obtained, which is reacted further without further purification.

(5) 2.5 ml of piperidine are added to a suspension of the resin “CD” in 10 ml of DMF to remove the FMbC protective group and the mixture is stirred for 30 min. It is then filtered and the resin is washed with DMF and dichloromethane and dried.

After taking up the resin again in 5 ml of DMF and addition of 0.2 ml of DIC, 0.6 g of 4-(1H-benzimidazol-2-ylamino)butanoic acid and 20 mg of DMAP, it is stirred for 15 h, filtered and washed. For cleavage, the resin is treated with 0.5 ml of 4N NaOH, 1 ml of methanol and 4 ml of dioxane. The cleavage solution is neutralized and worked up in the customary manner. 3-{2-[4-(1H-Benzimidazol-2-ylamino)butyrylamino]acetyl-amino}-3-(4′-chlorobiphenyl-4-yl)propionic acid is obtained.

3-{2-[4-(1H-Benzimidazol-2-ylamino)butyrylamino]acetyl-amino}-3-(4′-chlorobiphenyl-4-yl)propionic acid trifluoroacetate is obtained by preparative HPLC, RT 9.81 min, FAB-MS (M+H)⁺ 535.

### EXAMPLE 2

Analogously to Example 1, the resin “AB” is reacted with 3-chloro-4-fluorophenylboronic acid, then with FMOC-protected glycine and 4-(1H-benzimidazol-2-yl-amino)butanoic acid. 3-{2-[4-(1H-Benzimidazol-2-yl-amino)butyrylamino]acetylamino}-3-(31-chloro-4′-fluorobiphenyl-4-yl)propionic acid is obtained.

3-{2-[4-(1H-Benzimidazol-2-ylamino)butyrylamino]acetyl-amino}-3-(3′-chloro-4′-fluorobiphenyl-4-yl)propionic acid trifluoroacetate is obtained by preparative HPLC, RT 8.91 min, FAB-MS (M+H)⁺ 552.

Analogously to Example 1, the resin “AB” is reacted with 3-fluorophenylboronic acid, then with FMOC-protected glycine and 4-(1H-benzimidazol-2-ylamino)-butanoic acid. 3-{2-[4-(1H-Benzimidazol-2-ylamino)-butyrylamino]acetylamino}-3-(3′-fluorobiphenyl-4-yl)-propionic acid is obtained.

3-{2-[4-(1H-Benzimidazol-2-ylamino)butyrylamino]acetyl-amino}-3-(3′-fluorobiphenyl-4-yl)propionic acid trifluoroacetate is obtained by preparative HPLC, RT 9.65 min, FAB-MS (M+H)⁺ 518.

### EXAMPLE 3

Analogously to Example 1, the resin “DE” [prepared by reaction of 3-(3-bromophenyl)-3-tert-butoxycarbonyl-aminopropionic acid with the solid phase polystyrene A OH (Rapp, item No. HA 1 400 00)]

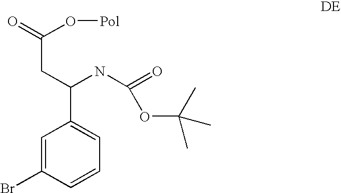

is reacted with 3-chloro-4-fluorophenylboronic acid, then with FMOC-protected glycine and 4-(1H-benzimidazol-2-ylamino)butanoic acid. 3-{2-[4-(1H-Benzimidazol-2-ylamino)butyrylamino]acetylamino}-3-{3′-chloro-4′-fluorobiphenyl-3-yl)propionic acid is obtained.

3-(2-[4-(1H-Benzimidazol-2-ylamino)butyrylamino]acetyl-amino}-3-(3′-chloro-4′-fluorobiphenyl-3-yl)propionic acid trifluoroacetate is obtained by preparative HPLC.

Analogously to Example 1, the resin “DE” is reacted with 3-fluorophenylboronic acid, then with FMOC-protected glycine and 4-(1H-benzimidazol-2-ylamino)-butanoic acid. 3′-{2-[4-(1H-Benzimidazol-2-ylamino)-butyrylaminolacetylamino}-3-(31-fluorobiphenyl-3-yl)-propionic acid is obtained.

3-{2-[4-(1H-Benzimidazol-2-ylamino)butyrylamino]acetyl-amino}-3-(3′-fluorobiphenyl-3-yl)propionic acid trifluoroacetate is obtained by preparative HPLC.

### EXAMPLE 4

Analogously to Example 1, the resin “AB” is, reacted with 4-ethoxyphenylboronic acid, then with FMOC-protected glycine and 4-(1H-imidazol-2-ylamino)butanoic acid. 3-(4′-Ethoxybiphenyl-4-yl)-3-{2-[4-(1H-imidazol-2-ylamino)butyrylamino]acetylamino}propionic acid is obtained.

3-(4′-Ethoxybiphenyl-4-yl)-3-{2-[4-(1H-imidazol-2-ylamino)butyrylamino]acetylamino}propionic acid trifluoroacetate is obtained by preparative HPLC.

Analogously to Example 1, the resin “id” is reacted with 3-cyanophenylboronic acid, then with FMOC-protected glycine and 4-(4,5-dihydro-1H-imidazol-2-ylamino)butanoic acid. 3-(3′-Cyanobiphenyl-4-yl)-3-{2-[4-(4,5-dihydro-1H-imidazol-2-ylamino)butyrylamino]-acetylamino}propionic acid is obtained.

3-(3′-Cyanobiphenyl-4-yl)-3-{2-[4-(4,5-dihydro-1H-imidazol-2-ylamino)butyrylamino]acetylamino}propionic acid trifluoroacetate is obtained by preparative HPLC.

Analogously to Example 1, the resin “AB” is reacted with 4-chlorophenylboronic acid, then with FMOC-protected glycine and 4-(4,5-dihydro-5-oxo-1H-imidazol-2-ylamino)butanoic acid. 3-(4′-Chlorobiphenyl-4-yl)-3-{2-(4-(5-oxo-4,5-dihydro-1H-imidazol-2-ylamino)-butyrylamino]acetylamino}propionic acid is obtained.

3-(41-Chlorobiphenyl-4-yl)-3-{2-[4-(5-oxo-4,5-dihydro-1H-imidazol-2-ylamino)butyrylamino]acetylamino}-propionic acid trifluoroacetate is obtained by preparative HPLC.

The following examples relate to pharmaceutical preparations:

### EXAMPLE A

### Injection vials

A solution of 100 g of an active compound of the formula I and 5 g of disodium hydrogenphosphate is adjusted to pH 6.5 in 3 l of double-distilled water using 2n hydrochloric acid, sterile-filtered dispensed into injection vials, lyophilized under sterile conditions and aseptically sealed. Each injection vial contains 5 mg of active compound.

### EXAMPLE B

### Suppositories

A mixture of 20 g of an active compound of the formula I is fused with 100 g of soya lecithin and 1400 g of cocoa butter, poured into moulds and allowed to cool. Each suppository contains 20 mg of active compound.

### EXAMPLE C

### Solution

A solution is prepared from 1 g of an active compound of the formula I, 9.38 g of NaH₂PO₄.2H₂O, 28.48 g of Na₂HPO₄-12H₂O and 0.1 g of benzalkonium chloride in 940 ml of double-distilled water. The solution is adjusted to pH 6.8, made up to 1 l and sterilized by irradiation. This solution can be used in the form of eye drops.

### EXAMPLE D

### Ointment

500 mg of an active compound of the formula I are mixed with 99.5 g of petroleum jelly under aseptic conditions.

### EXAMPLE E

### Tablets

A mixture of 1 kg of active compound of the formula I, 4 kg of lactose, 1.2 kg of potato starch, 0.2 kg of talc and 0.1 kg of magnesium stearate is compressed in the customary manner to give tablets such that each tablet contains 10 mg of active compound.

### EXAMPLE F

### Coated tablets

Tablets are pressed analogously to Example E and are then coated in the customary manner with a coating of sucrose, potato starch, talc, tragacanth and colourant.

### EXAMPLE G

### Capsules

2 kg of active compound of the formula I are filled into hard gelatin capsules in the customary manner such that each capsule contains 20 mg of the active compound.

### EXAMPLE H

### Ampoules

A solution of 1 kg of active compound of the formula I in 60 l of double-distilled water is sterile-filtered, dispensed into ampoules, lyophilized under sterile conditions and aseptically sealed. Each ampoule contains 10 mg of active compound.

### EXAMPLE I

### Inhalation Spray

14 g of active compound of the formula I are dissolved in 10 l of isotonic NaCl solution and the solution is filled into commercially available spray containers having a pump mechanism. The solution can be sprayed into the mouth or nose. One puff of spray (approximately 0.1 ml) corresponds to a dose of approximately 0.14 mg.

1. Compounds of the formula I 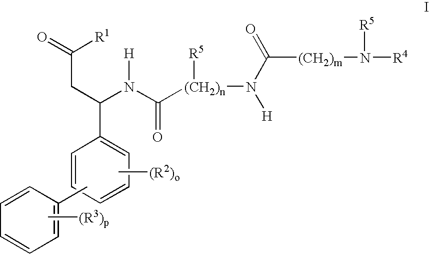
in which
R¹ is OR or N(R)₂,
R is H, A, cycloalkyl, Ar, arylalkyl or Pol,
R² and R³ in each case independently of one another are H, A, Hal, NO₂, OR, N(R)₂, CN, CO—R, SO₃R, SO₂R, NH—C(O)A or SR,
R⁴ is a mono- or bicyclic aromatic heterocycle having 1 to 4 N atoms, which can be mono- or disubstituted by Hal, R, OR, CN, N(R⁵ )₂ or NO₂, where pyridine, pyridazine, pyrimidine, pyrazine, 1,3,5- 1,2,4- and 1,2,3-triazine and tetrazine are excluded,
R⁵ is H of A,
R⁶ is Hal or NO₂,
A is alkyl having 1 to 8 C atoms, where the alkyl groups can be mono- or polysubstituted by R⁶ and/or their alkyl carbon chain can be interrupted by —O—,
Ar is aryl which is unsubstitited or mono-, di- or trisubstituted,
cycloalkyl is cycloalkyl having 3 to 15 C atoms,
Hal is F, Cl, Br or I,
Pol is a solid phase without a terminal functional group,
n, m in each case independently of one another are 1, 2, 3, 4, 5 or 6,
o is 1, 2, 3 or 4, and
p is 1, 2, 3, 4 or 5,
and their physiologically acceptable salts and solvates.

2. Compounds according to claim 1
a) 3-{2-[4-(1H-Benzimidazol-2-ylamino)butyryl-amino]acetylamino}-3-(4′-chlorobiphenyl-4-yl)-propionic acid,
b) 3-{2-[4-(1H-Benzimidazol-2-ylamino)butyryl-amino]acetylamino}-3-(31-chloro-4′-fluorobiphenyl-4-yl)-propionic acid,
c) 3-{2-[4-(1H-Benzimidazol-2-ylamino)butyryl-amino]acetylamino}-3-(3′-fluorobiphenyl-4-yl)-propionic acid,
and their physiologically acceptable salts and solvates.

3. Process for the preparation of the compounds of the formula I according to claim 1 and their salts and solvates, characterized in that
(a) a compound of the formula II 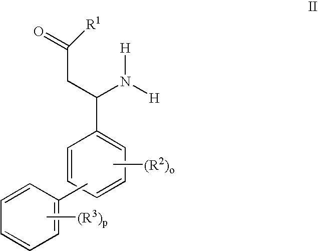
in which R, R¹, R², R³, o and p have the meanings indicated in claim 1, but R≠H and in which free hydroxyl or amino groups as substituents R² or R³ are present protected by protective groups,
is reacted with a compound of the formula III 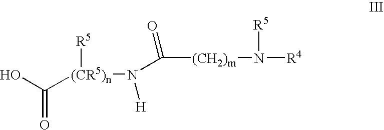
in which R⁴, R⁵, n and m have the meanings indicated in claim 1
and, if appropriate, the radical R≠H is converted into the radical R=H and the protective groups on R² and/or R³ are removed, or
(b) a compound of the formula IV 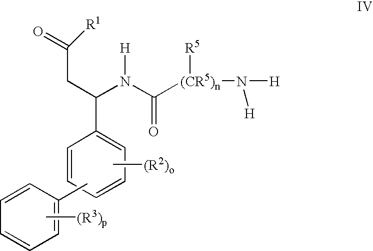
in which R, R¹, R², R³, R⁵, n; o and p have the meanings indicated in claim 1, but R≠H and in which free hydroxyl or amino groups as substituents R² or R³ are present protected by protective groups,
is reacted with a compound of the formula V 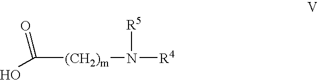
in which R⁴, R⁵ and m have the meanings indicated in claim 1
and, if appropriate, the radical R≠H is converted into the radical R=H and the protective groups on R² and/or R³ are removed, or
(c) in a compound of the formula I, one or more radicals R, R¹, R², R³, R⁴ and/or R⁵ are converted into one or more radicals R, R¹, R², R³, R⁴ and/or R⁵;
by, for example,
vii) alkylating a hydroxyl group,
viii) hydrolysing an ester group to a carboxyl group,
ix) esterifying a carboxyl group,
x) alkylating an amino group,
xi) reacting an aryl bromide or iodide by means of a Suzuki coupling with boronic acids to give the corresponding coupling products, or
xii) acylating an amino group, and/or
converting a basic or acidic compound of the formula I into one of its salts or solvates by treating with an acid or base.

4. Compounds of the formula I according to claim 1 and their physiologically acceptable salts or solvates as pharmaceutical active compounds.

5. Compounds of the formula I according to claim 1 and their physiologically acceptable salts or solvates as integrin inhibitors.

6. Compounds of the formula I according to claim 1 and their physiologically acceptable salts or solvates for use in the control of diseases.

7. Pharmaceutical preparation, characterized in that it contains at least one compound of the formula I according to claim 1 and/or one of its physiologically acceptable salts or solvates.

8. Use of compounds of the formula I according to claim 1 and/or their physiologically acceptable salts or solvates for the production of a medicament.

9. Use of compounds of the formula I according to claim 1 and/or their physiologically acceptable salts or solvates for the production of a medicament for the control of thromboses, cardiac infarcts, coronary heart diseases, arteriosclerosis, inflammation, tumours, osteoporosis, infections, rheumatoid arthritis, diabetic retinopathy and restenosifs after angioplasty.

10. Use of compounds of the formula I according to claim 1 and/or their physiologically acceptable salts or solvates in pathological processes which are supported or propagated by angiogenesis.
